(19) United States
(12) Patent Application Publication
(10) Pub. No.: US 2023/0233279 A1
(43) Pub. Date: Jul. 27, 2023

(54) PACKAGING FOR SURGICAL IMPLANT

(71) Applicant: Neurogami Medical, Inc., Mountain View, CA (US)

(72) Inventors: Stephen J. Hebert, San Francisco, CA (US); Bartosz Bojanowski, San Francisco, CA (US)

(73) Assignee: Neurogami Medical, Inc., Mountain View, CA (US)

(21) Appl. No.: 18/091,462

(22) Filed: Dec. 30, 2022

Related U.S. Application Data
(60) Provisional application No. 63/301,551, filed on Jan. 21, 2022.

(51) Int. Cl. A61B 50/30; A61B 17/12

(52) U.S. Cl. CPC A61B 50/30; A61B 17/1214; A61B 17/12113; A61B 17/12163; A61B 2017/1205; A61B 2050/3008; A61B 2050/3015

(57) ABSTRACT

A packaging for a vascular implant having a first packaging tube and a holder having at least one recess. The implant is positioned within the holder in a first looped condition, wherein the implant is movable from the holder into the first packaging tube and maintained within the first packaging tube in a second more linear condition. A looped portion of the implant includes a plurality of loops receivable in the at least one recess.

### BACKGROUND

This application claims priority from provisional application serial no. 63/301,551, filed Jan. 21, 2022, the entire contents of which are incorporated herein by reference.

### TECHNICAL FIELD

This application relates to packaging for surgical implants.

### BACKGROUND OF RELATED ART

An aneurysm is a localized, blood-filled balloon-like bulge that can occur in the wall of any blood vessel, as well as within the heart. There are various treatments for aneurysms. One endovascular treatment option for aneurysms is complete reconstruction of the damaged vessel using a vascular prosthesis or stent-graft. However, stent-grafts are not a treatment option for intracranial aneurysms due to the risk of cutting off blood flow to feeder vessels that may be vital for brain function. Stent-grafts can also be stiff, hard to deliver/retract, and can be highly thrombogenic within the parent vessel, all of which are undesirable features for intracranial aneurysm treatment.

As a result, endovascular treatment of intracranial aneurysms has centered on packing or filling an aneurysm with material or devices in order to achieve a high packing density to eliminate circulation of blood, which leads to thrombus formation and aneurysm closure over time.

Various types of current mechanical vaso-occlusive devices are composed of metals or alloys, and biocompatible fibers, for example. Generally, the materials are formed into tubular structures such as helical coils of shape memory which can have secondary shapes. Materials can also be formed into tubes/strings/braided sutures, cables or braids. Metal coils can also be covered by winding on thrombogenic fiber. Braided or polymer coils can be non-expandable or self-expandable devices. These devices can be made from materials such as textiles, polymers, metal or composites using known weaving, knitting, and braiding techniques and equipment. Included in the weave or the finished braid can be optional mono or multifilament fiber manufactured to impart additional features or effects (e.g., radiopacity and thrombogenicity).

Such devices can be formed into secondary shapes, such as helical coils that have a 3D helical or corkscrew secondary shape, they can be dimensioned to engage the walls of the aneurysm, or they can have other shapes (e.g., random, “flower”, or three dimensional complex shapes). Braided devices can be shaped into these secondary shapes while having tubular cross sections either that self-expand or maintain their shape/size.

Non-expandable braids can also cover core or primary structures, such as coils or other braids. Much like the above braid structures, these covers may have open cell designs (e.g., inner coil structure is visible through the braid) or closed cell designs.

Regardless of configuration, it is difficult to achieve high packing densities and rapid flow stagnation with traditional metal coils. If an aneurysm sac is not sufficiently packed to stop or slow blood flow, any flow through the neck of the aneurysm may prevent stasis or cause coil compaction, leading to recanalization of the aneurysm. Conversely, tight packing of metal coils in large or giant aneurysms may cause increased mass effect (compression of nearby tissue and stretching of aneurysm sac) on adjacent brain parenchyma and cranial nerves. Coil prolapse or migration into parent vessels is another possible issue with non-expanding devices, especially in wide neck aneurysms.

A major problem for self-expanding braid designs is sizing. The implant has to be accurately sized so that upon expansion it occupies enough volume to fill the entire aneurysm, dome to neck. Undersized devices lead to insufficient packing as described above, whereas oversizing risks rupturing the aneurysm or blockage of parent vessel.

While the above devices attempted to treat intracranial aneurysms with minimally invasive techniques, there was still a need for a highly compliant and thrombogenic filler that blocks blood flow within the sac of the aneurysm without the foregoing drawbacks. The micrografts of commonly assigned U.S. Pat. Nos. 9,999,413, 10,736,730, 10,857,012, and 10,925,611 met this need. The patents disclose devices that advantageously achieve sufficient flexibility to enable advancement through the tortuous vasculature into the cerebral vasculature and high packing densities while maintaining a high concentration of thrombogenic material. These devices also cause rapid clotting of the blood and promote tissue ingrowth within a relatively short period of time. The devices are soft, compressible and absorbent to retain blood. These devices are designed for minimally invasive insertion, and are easy to deliver and deploy at the intracranial site as well as manufacturable in a small enough size for use in cerebral vasculature. That is, these devices are constructed to effectively pack the aneurysm without damaging the sac or other tissue while promoting rapid clotting and healing of an intracranial aneurysm with reduction in mass effect.

Delivery of the implants of these patents as well as other types of implants described above can be challenging, especially with implants having a secondary shape that is helical/coiled or complex/3D. Further, the implants of these patents as well as the other types of implants described above incorporate polymeric materials and have secondary shapes whereby if packaged and stored in a straightened shape, might not fully return to their secondary shape when implanted.

The inventors of commonly assigned U.S. Pat. No. 10,925,611, the entire contents of which are incorporated herein by reference, attempted to solve the problems associated with packaging and storage of implants. Although providing a solution, the same inventors discovered room for further improvement of the packaging to facilitate storage and reduce the chances of the implant coils tangling during storage.

### SUMMARY OF INVENTION

The inventors of U.S. Pat. No. 10,925,611 developed packaging for surgical implants which ensured their secondary shape was maintained during implantation. The same inventors, as disclosed in the inventions herein, developed an improved packaging and storage system for implants which maintains their secondary shape and reduces tangling during storage and in preferred embodiments allows the user to visualize the 3D shape of implant prior to use, thereby further facilitating deployment and usability of the implant in the vasculature. This is achieved in the present invention by packaging having one or more compartments for retaining loops of the implant. The present invention also provides for better organization of the packaging tubes.

In accordance with one aspect of the present invention, a packaging for a vascular implant is provided comprising a first packaging tube and a holder having at least one recess wherein the implant is positioned within the holder in a first looped condition. A looped portion of the implant includes a plurality of loops receivable in the at least one recess. The implant is movable from the holder into the first packaging tube and maintained within the first packaging tube in a second more linear condition.

In some embodiments, the packaging further comprises a delivery sheath having a lumen and a delivery member positioned within the lumen, the delivery sheath positioned within the first packaging tube (hoop), and the implant is pulled into the delivery sheath by the delivery member to be maintained within the delivery sheath within the first packaging tube. The packaging in some embodiments includes a second packaging tube (hoop) having a first end and a second opposite end, and the first packaging tube has a first end adjacent to the holder and a second opposite end, wherein the first end of the second packaging tube is spaced from the second end of the first packaging tube to form a gap between the first and second packaging tubes.

In some embodiments, the holder includes at least one arcuate channel to receive the first packaging tube. In some embodiments, the at least one recess comprises a plurality of spaced apart recesses. In some embodiments, the plurality of recesses are longitudinally aligned. In some embodiments, each of the plurality of recesses is a same size; in other embodiments, at least one of the plurality of recesses is a different size than another of the plurality of recesses.

In some embodiments, lateral movement of the sheath is constrained within the holder. In some embodiments, the sheath is constrained by a friction element.

In accordance with another aspect of the present invention, a packaging for a vascular implant is provided comprising a container having a) a plurality of recesses each dimensioned to receive a looped portion of the implant, b) a channel, and c) a first member extending within the channel and engageable with an end region of the implant.

In some embodiments, the container comprises an arcuate channel to receive a portion of a packaging tube into which the implant is subsequently pulled. In some embodiments, the packaging tube receives the first member in a lumen therein.

In some embodiments, the recesses have covers to form bulbs. In some embodiments, the bulbs are transparent.

The packaging in some embodiments includes a first packaging tube and a second packaging tube, a first end of the second packaging tube is spaced from the second end of the first packaging tube to create a gap between the first and second packaging tubes, wherein the exposed portion of the delivery member and the exposed portion of the delivery sheath are exposed within the gap between the first and second packaging tubes. In some embodiments, the delivery member extends into the second packaging tube and a proximal end of the delivery sheath terminates in the gap between the first and second packaging tubes. In some embodiments, application of a pulling force to the delivery member relative to the delivery sheath pulls the vascular implant from the container where it is held in an unconstrained condition into the delivery sheath so the vascular implant has a reduced transverse dimension as it straightens as it is pulled into the sheath.

### BRIEF DESCRIPTION OF THE DRAWINGS

So that those having ordinary skill in the art to which the subject invention appertains will more readily understand how to make and use the surgical apparatus disclosed herein, preferred embodiments thereof will be described in detail hereinbelow with reference to the drawings, wherein:

FIG. 1 is a top view of the packaging for the vascular implant in accordance with one embodiment of the present invention;

FIG. 2 is a view similar to FIG. 1 showing the long packaging tube highlighted for clarity and the delivery wire extending from the tubes;

FIG. 3 is a bottom perspective view of the container of the packaging of FIG. 1;

FIG. 4 is an exploded view of the container of FIG. 3;

FIG. 5 is top perspective view of the container of the packaging of FIG. 1;

FIG. 6 is a top view of the container of the packaging of FIG. 1 showing the implant retained within the bulbs of the container;

FIGS. 7-12 illustrate the steps of removing the vascular implant from the packaging of FIG. 1 wherein

FIG. 7 illustrates the delivery wire being pulled proximally (in the direction of the arrow) relative to the delivery sheath to pull the implant into the sheath, the implant shown partially pulled within the sheath;

FIG. 8 illustrates the delivery wire being pulled further proximally relative to the delivery sheath, the implant shown removed from the container and fully within the sheath;

FIG. 9 illustrates grasping of the sheath and delivery wire for proximal movement in the direction of the arrow;

FIG. 10 illustrates the sheath and wire being pulled in tandem proximally to pull the sheath and delivery wire (delivery system) out of the packaging tube (hoop);

FIG. 11 illustrates grasping of the delivery sheath and delivery wire for movement in the direction of the arrow to remove the delivery wire from the second packaging tube (hoop); and

FIG. 12 illustrates the delivery wire pulled distally out of the second packaging hoop.

### DETAILED DESCRIPTION

The present invention provides packaging for micrografts (implants) described in detail in commonly assigned U.S. Pat. No. 10,925,611 (hereinafter the ‘611 patent), the entire contents of which are incorporated herein by reference. Initially, a brief discussion of the micrograft is provided, followed by a discussion of the packaging of the present invention. Note that further details of the implant, and alternate embodiments thereof, are discussed in detail in the ‘611 patent and in U.S. Pat. No. 10,857,012 (hereinafter the ‘012 patent), the entire contents of which are incorporated herein by reference.

It should be appreciated that the packaging of the present invention can be used for implants other than those described in the ‘611 and ‘012 patent.

FIGS. 4F-4M of the ‘012 patent show views of one embodiment of an intra-aneurysmal micrograft for insertion into an intracranial aneurysm. The micrograft has a biocompatible non-self-expandable absorbent braided polymeric textile tubular body that is crimped to reduce stiffness and increase wall thickness and fabric density. The micrograft has sufficient stiffness as well as sufficient flexibility. It further is structured to enable a triple capillary action to promote blood clotting. The micrograft further preferably has a high surface area for increased blood absorption, is radially deformable, has a low friction surface for ease of delivery and can be shape set to enhance packing of the aneurysm. The micrograft is especially designed to induce blood stagnation or clot to rapidly treat the aneurysm. The micrograft is configured for delivery to an intracranial aneurysm, although it can be utilized for occlusion in aneurysms in other areas of the body as well as for occlusion in other vascular regions or in non-vascular regions.

The micrograft is constructed of multi-filament interlaced yarns, wherein each yarn is composed of a plurality of polyester filaments having pores or spaces therebetween, and the plurality of yarns also have pores or spaces therebetween. Blood flows through the micrograft in a distal to proximal direction. The micrograft can be navigated to and into the cranial vasculature for placement within a cranial vessel.

Each of the multi-filament yarns are made of multiple wettable micro-filaments, or fibers, assembled with spaces (pores) between them. The pores are sufficiently sized to induce capillary action when contacted by a liquid, resulting in the spontaneous flow of the liquid along the porous yarn (i.e., wicking). This capillarity between fibers (intra-fiber) within the yarn is termed as “micro-capillary” action. As a result, a sufficiently wettable and porous yarn has high wickability and transport liquid along its length. The multiple filaments also provide a high surface area and can be hydrophilic or hydrophobic.

This assembly of the two or more wickable multi-filament yarns into a permeable structure (such as a textile) results in a “macro-capillary” action, i.e., the transporting of liquid between the yarns and throughout the structure.

The multi-filament yarns can be assembled into a textile tubular structure using a braider or other textile manufacturing equipment and methods.

The vascular graft (micrograft) has a proximal opening at the proximal end and a distal opening at the distal end for blood flow into the distal end and through the lumen (the proximal and distal openings aligned with a longitudinal axis), thereby forming a conduit for transport of blood through the continuous inside lumen (inside diameter). A capillary effect is created within the vascular graft when the biocompatible structure is exposed to blood such that blood is transported in a proximal direction through the distal opening in the vascular graft and through the vascular graft wherein blood clots. Thus, blood initially flows through the distal opening, through the vascular graft and towards the proximal opening, with blood quickly stagnating within the graft. In some instances, blood will exit the proximal opening (e.g., if there is sufficient pressure); in other instances, capillary action will only fill the graft and not cause flow out the proximal opening. In other instances, the proximal end does not have a proximal opening. The vascular graft retains blood, and becomes saturated with blood, to promote clotting. The outer member, i.e., the textile structure, is configured as a tubular member for flow therein, functioning as a capillary tube. The tubular textile member is configured in a closed cell fashion so as to form a tube for flow therethrough, i.e., the lumen inside the textile structure is sufficiently small to enable function as a capillary tube, but the textile structure still has sufficient sized openings/spaces for absorbing blood through and along the yarns and filaments as described herein. Thus, a continuous wall (continuous inner diameter) is formed along the length of the textile structure to retain blood while also maintaining small spaces (micro-capillaries) in between fibers to wick and absorb blood. This non-expanding closed cell or tight textile, e.g., braided, structure is maintained since the diameter of the textile structure (and thus the diameter of the vascular graft) does not change from the delivery to implant positions.

The tubular textile structure (which forms a braid in some embodiments) forms a continuous circumferential wall along a length without large spaces between the filaments and/or yarns. This continuous wall is shown in the tight spacing of FIGS. 4A-4D of the ‘611 patent and thus creates a continuous outer member (low porosity wall) to contain and direct flow. The yarns of the textile structure are close enough to form a continuous wall to wick and transfer blood via the wall and inside lumen.

The capillary spaces formed between yarns are termed macro-capillary and capillary spaces formed between individual fibers of a yarn is termed micro-capillary. The capillary action occurs as the yarns making up the wall of the textile structure and the fibers making up the yarns are assembled close enough, as shown in FIGS. 4A and 4M of the ‘611 patent, to create micro-capillaries that induce wicking. Thus, the tubular textile structure utilizes the three capillary actions (i.e., inside (inner) lumen, inter-yarn and inter-filament capillary actions) to act as a capillary tube and also achieves blood retention inside the tubular structure.

By forming the textile structure as a tubular member (rather than winding/braiding the filaments about an inner element), and then inserting/positioning the inner core element therein for attachment to the outer textile structure, portions of the inner surface of the inner wall of the textile structure are in contact with the inner element.

The micrograft includes a permanent core element formed of a metal coil having a lumen therein.

Due to the manufactured tube’s relatively small inner diameter and a sufficiently dense interlacing braid pattern (i.e., a filamentary wall structure with sufficiently small pore size such that it retains fluid), the third capillary effect is created. When properly sized, this third capillary effect is responsible for spontaneous flow of liquid inside the micrograft lumen, e.g., within the lumen of the braid, in a proximal direction.

To reduce stiffness to assist delivery and packing of the aneurysmal sac, the micrograft tubular body (braid) is crimped during manufacture, i.e., longitudinally compressed and heat set. As the braid is compressed, axial orientation of the braided strands is reduced thereby increasing braid angle with respect to the longitudinal axis of the tubular body which reduces their influence on overall stiffness of the structure, much like a straight wire taking on a more flexible form when coiled. Crimping also effectively increases the PPI, wall thickness, and linear density of the braid by axially compressing the structure and filament bundles. This compression causes an outward radial expansion and an increase in wall thickness of the tube. The resulting braid is much more deflectable, has reduced bend radius, a higher density and up to 2x to 3x or higher increase in PPI, depending on braid structure and compressive force applied.

This axial compression also causes the braid structure to “snake” or produce a spiral wavy forming a series of macro peaks and valleys, termed “macro-crimps”, in a sinusoidal shape.

FIGS. 4A, 4B, 4D and 4E-4M of the ‘611 patent show an embodiment of the micrograft having a core element having a lumen for blood flow in the aforementioned capillary effect. The core element is a coil and the lumen extends through the coil from the proximal end to the distal end. The coil can be composed of a metal such as platinum or a platinum tungsten alloy. In manufacture, the textile structure in the form of a tubular braid is positioned over the coil, and the braid is formed separately into a tubular shape with a lumen or longitudinally extending opening extending from the proximal end to the distal end for receipt of the coil. The braid is preferably composed of PET or other thrombogenic material and is preferably substantially a closed cell design to provide a large percentage of outer surface area for contact with the blood and/or vessel/aneurysm wall, but has spaces between the yarns and filaments to enable blood flow into and/or through the device to achieve the capillary effects. The micrograft, with the braid and attached inner coil, is formed into a helical coil shape as shown in FIG. 4K of the ‘611 patent with a lumen extending along its length.

This configuration of the embodiment of FIG. 4A of the ‘611 patent also encourages rapid blood clotting and in some instances clotting can occur immediately upon implantation. When the micrograft (implant) is held in the delivery system within the vessel/aneurysm but prior to release from the delivery system, the micrograft becomes filled partially or entirely with blood so that blood stagnation can commence even before the micrograft is released and implanted, thereby expediting thrombus formation. Saturation of the micrograft in the delivery assembly and once implanted accelerates and/or improves thrombosis.

Note the braid fibers are not only thrombogenic (attract blood platelets and proteins which promote clot) due to their material, e.g., PET can be used as the filaments or as a thrombogenic surface, but also promote stasis as the braid structure traps blood.

A tube, preferably composed of Nitinol, is seated within proximal coils of the helical core element (coil), screwed or twisted into the proximal coil windings of the helical core element to provide structure for engagement with a delivery device. The braid is melted onto the tube, and the tube extends proximally of the core element. It also extends proximally of the tubular textile structure so a proximal region is exposed for engagement by a delivery member. A distal portion of the tube is within the tubular textile structure.

As noted above, the braid of the implant is preferably non-expandable. That is, after formed, a dimension measured through a transverse cross-section of the implant (braid and coil) is the same in a delivery position within a delivery member as in the placement position. The implant, however, may be stretched or straightened to a reduced profile position for delivery and then released for placement to assume its coil shape discussed above. However, when it moves from the delivery to the placement position, the braid does not expand. The change is to the implant (braid and coil) from the linear shape within the delivery member to its secondary helical shape within the body, but the combined thickness of the braid and coil (i.e., the outer diameter of the braid) remains constant during delivery and placement. This is in contrast with expandable braids wherein the diameter of the braid increases when exposed from the delivery member and in the placement position.

The implant of the ‘611 patent, as described, can be shape set into any complex three dimensional configuration including a cloverleaf, a figure-8, a flower-shape, a vortex-shape, an ovoid, randomly shaped, substantially spherical shape, etc. The soft open pitch coil within the braid aids in visualization. If stiffness of such metal coil is sufficiently low, the secondary shape-set of the polymer braid will drive the overall shape of the device. In other words, the secondary shape of the braid molds the unshaped metal coil which normally shape sets at temperatures much greater than the glass transition temperature of polymers.

The implant is preset to a non-linear configuration and advanced to the aneurysm in a substantially linear configuration and then returns to the same non-linear configuration or different non-linear configuration when delivered into the aneurysm, depending on the space within the aneurysm.

As the micrograft is deployed into the aneurysm, it will take on any preset secondary shapes and random shapes due to contact with the aneurysm walls.

As discussed in the ‘611 patent, the delivery wire for the implant can be a guidewire. Therefore, if desired, the micrograft delivery system with guidewire can be loaded into the microcatheter prior to catheter placement. The entire assembly, microcatheter and micrograft delivery system, can then be tracked to the aneurysm site using the delivery system’s guidewire as the primary tracking wire. Alternately, as described, the guidewire and microcatheter can be tracked to the aneurysm site and a rapid exchange catheter can be advanced subsequently. The micrograft can alternatively be constructed to mate with other microcoil delivery systems that provide a timed and controlled release, e.g., electrolytic detachment.

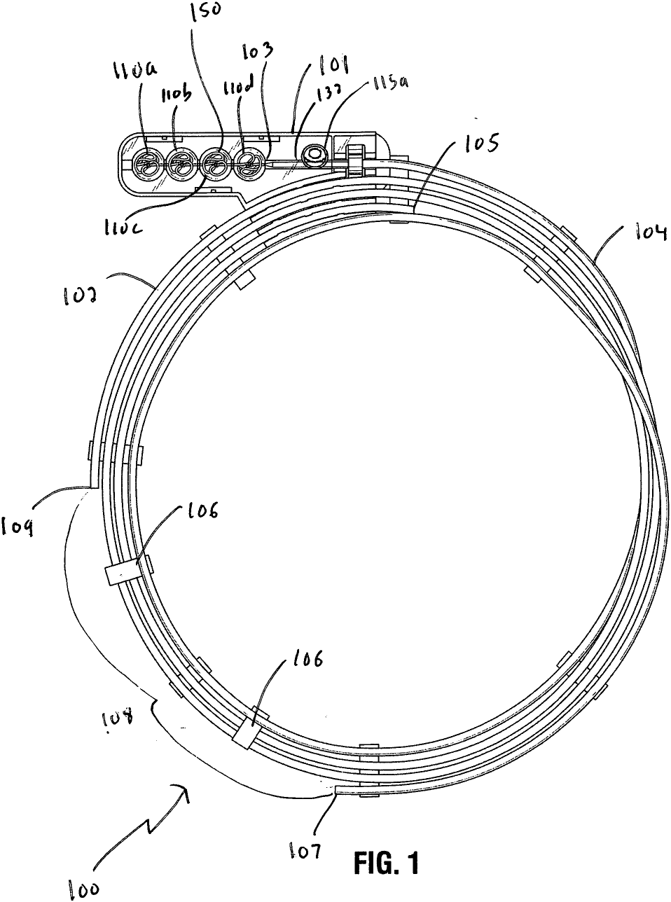

Turning now to the packaging of the present invention, FIGS. 1-12 illustrate one embodiment of packaging for the vascular implants disclosed in the ‘611 patent, however, the packaging of the present invention can also be used for other implants as well such as shape memory implants. The vascular implant shown has a secondary helical shape like the vascular implant of FIG. 4K of the ‘611 patent with a series of loops. The implant has a proximal tube engaged by the delivery element as in FIG. 39 of the ‘611 patent.

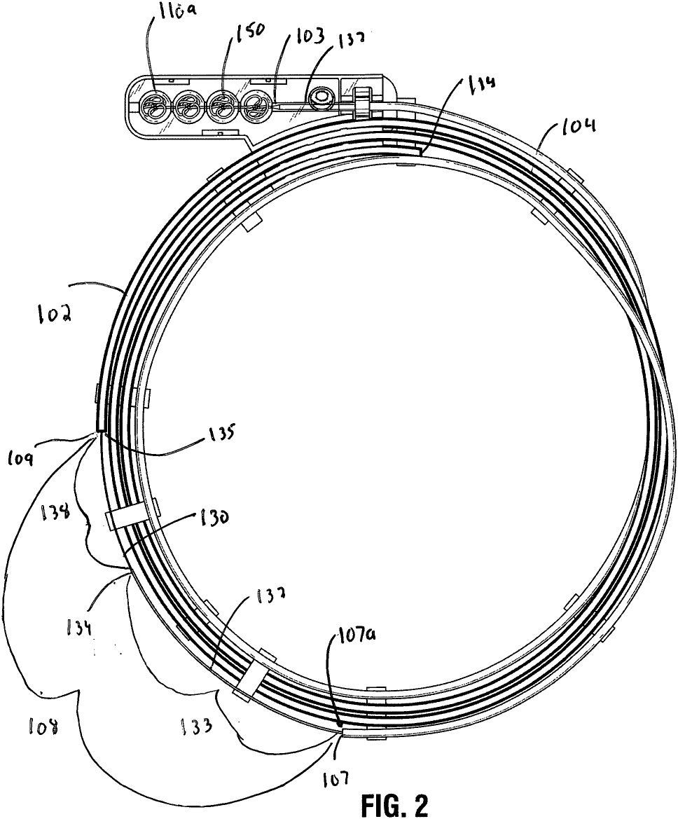

The packaging is designated generally by reference numeral 100 and is contained in a shipping pouch or package (not shown). The packaging 100 includes container 101 (also referred to herein as the implant holder), a long packaging hoop 102 and a short packaging hoop 104. The hoops 102, 104 are in the form of tubes composed of a material such as HDPE or polypropylene that can be wrapped as shown without significant kinking that could inhibit movement of the components within the tubes 102, 104. The long packaging tube 102 is shown in FIG. 2 as darkened for ease of illustration but the long tube 102 can be of the same color as the short tube 104. A series of clips 106 spaced apart along the tubes retains the tubes (hoops) 102, 104 in the coiled (spiraled) configuration as shown, preferably evenly spaced, but different spacing is also contemplated.

Each of the tubes 102, 104 has a lumen extending therethrough. By way of example, the short tube 104 can have a length ranging from about 10 cm to about 100 cm, and more specifically about 70 cm; the long tube 102 can have a length ranging from about 100 cm to about 280 cm, and more specifically about 240 cm. Other lengths of the tubes 102, 104 are also contemplated. The long tube 102 is preferably longer than the short tube 104, however, in some embodiments they can have equal lengths or tube 102 can be shorter than tube 104. The ends of the tubes 102, 104 are spaced apart as shown to provide a gap 108.

More specifically, short tube 104 has a first distal end 103 adjacent the implant 150 and a second opposite proximal end 107. Long tube 102 has a proximal end 105 and an opposite distal end 109. The distal end 109 of the long tube 102 is spaced from the proximal end 107 of the short tube 104 to form the gap 108. This exposes the delivery member and delivery sheath (together referred to as the delivery system) as described below. The gap can be between about 2 cm and about 40 cm, and more specifically about 15 cm, although other dimensions are contemplated. The gap 108 can be considered/measured as the length of an arc extending between the proximal end 107 of short tube 104 and the distal end 109 of the long tube 102, equated with the length of the delivery system that is exposed between the tubes 102, 104. Note the term distal and proximal in reference to the packaging tubes 102 and 104 as used herein is used to identify the portion/ends in relation to the path of the implant from the container 101 through the tubes 102, 104 - the distal end/portion of the tube is adjacent where the path for the implant begins and the proximal end/portion is further from the start of the path of the implant i.e., the implant is first pulled through the distal end. Stated another away, the distal end of tube 104 is at the location of insertion of the implant from the container 101 into the tube 104.

The container 101 of packaging 100 has a series of bulbs 110a, 110b, 110c and 110d (collectively bulbs 110). The bulbs 110a-d are configured to hold a portion of the implant, e.g., loops of the implant to reduce the likelihood of tangling during storage and shipment. Additionally, the bulbs 110 allow easier/more effective sterilization of the implant since gas can permeate more easily especially if the implant is self expandable or has densely packed fibers. The bulbs 110 are shown longitudinally aligned but could be placed in other arrangements. Additionally, although four bulbs 110a 110b, 110c and 110d are shown, a different number of bulbs could alternatively be provided.

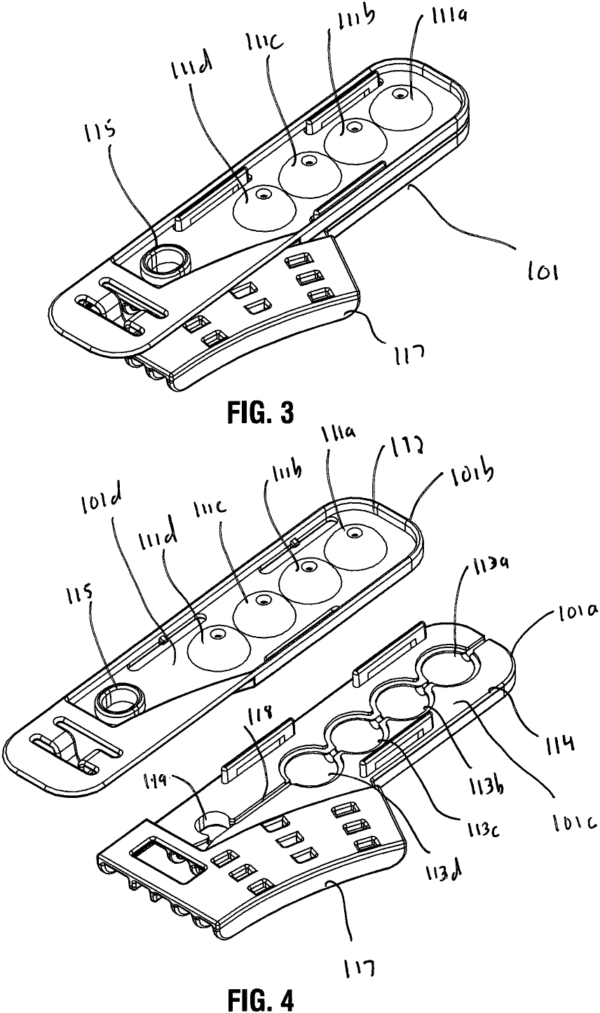

The container 101 is formed by the joining of two units (members/container portions) 101a and 101b as shown in FIG. 4. Member 101a is also referred to herein as the base and member 101b is also referred to herein as the cover. Container 101 is shown with base 101a and cover 101b separated in FIG. 4 and joined together as a unit in FIG. 3. These FIGS. 3 and 4 show a bottom perspective view of container 101.

Bulbs 110a-110d are spherical shaped, formed by the mating of the hemispherical domes 113a-113d (collectively domes 113) of base 101a and hemispherical domes 111a-111d (collectively domes 111) of cover 101b. That is, bulb 110a is formed by domes 111a and 113a; bulb 110b is formed by domes 111b and 113b; bulb 110c is formed by domes 111c and 113c; and bulb 110d is formed by domes 111d and 113d. As shown, each dome 113 has a circular or substantially circular recess on an internal surface 101c and a bulging dome on the opposing external surface. Each dome 111 has a circular or substantially circular recess on an internal surface and a bulging dome on an opposing external surface 101d. Note that other shapes for the bulbs/domes and/or recesses are also contemplated for storing the loops of the implant 150. In preferred embodiments, the bulbs 110 are transparent so the implant within the bulbs 110 can be visualized by the clinician so the implant size and shape can be determined prior to insertion into the body. In alternate embodiments, domes 111a-111d of cover 101b are transparent and the domes 113a-113d of base 101a are gray or colored to create a contrast with the implant to enhance visualization. Conversely, the domes 111a-111d of cover 101b could be gray or colored for contrast and the domes 113a-113d of base 101a transparent. The bulbs 110 reduce the likelihood of the implant tangling on itself in the packaging when compared to storing the implant in a single dome.

The bulbs in FIGS. 1-6 are shown as symmetric, e.g., the same size. Alternatively, the bulbs could be varied in size. For example, the distal bulb could be larger to accommodate the larger loops of the implant at the distal end. In some embodiments, two or more of the bulbs progressively decrease in size and decrease in a proximal direction to accommodate different size loops along the implant.

A narrow opening in the form of a channel 118 or a window extends from distalmost dome 113d to receptacle 119. Channel 118 also extends to join adjacent domes 113d, 113c, 113b and 113a. A similar channel may be provided on the inner surface of cover 101b to extend to receptacle 115 and to connect the domes 111a-111d. This channel 118 aids in loading of implant into the bulbs and prevents migration of the implant loops during storage and shipment.

Receptacles 115 and 119 receive transverse tube 115a (FIG. 1), or alternatively a foam or other structure can be utilized, which contacts and slightly presses against the delivery sheath to frictionally engage the sheath to restrict movement within the channel 118.

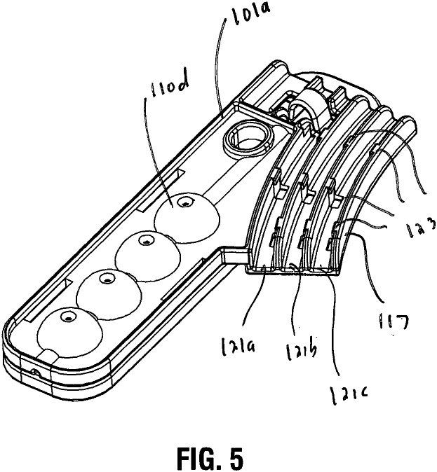

Base 101a also includes a curved channel 117. The channel 117, as shown in FIG. 5, includes three arcuate channels: an inner channel 121a, a middle channel 121b and an outer channel 121c (collectively curved channels 121). A fewer or greater number of curved channels could be provided. The outer channel 121c is furthest from the elongated linear channel 118. The curved channels 121 are each dimensioned to receive a portion of the long packaging tube 102 as it wraps (spirals) in a circular fashion. The curved channels 121 thus help organize the packaging tube 102 and constrain its spiral configuration. In some embodiments, the curved channels can receive a portion of the short packaging tube.

The curved channels 121 can each include locking tabs or divots 123, spaced apart and staggered on opposing walls, to provide an interference fit for the portions of the long tube 102 held therein. The channels 121 can also be sized for interference fit with the long tube portions. The number and placement of the locking tabs 123 can differ from that shown in FIG. 5 to perform the locking functions. Other locking structures could alternatively be provided. In some embodiments, the sizing of the arcuate channels itself retains the long tube 102 via a frictional/interference engagement.

The delivery wire 130 pulls the implant 150 out of the bulbs 110a-d and packaging container 101 as described in the method of use below.

In some embodiments, the container can be perforated or non-airtight to allow gas permeation and flow through the container during product sterilization, e.g., during ethylene oxide gas sterilization.

Referring back to FIGS. 1 and 2, the short tube 104 as shown forms a wrap of a little more than 360 degrees, e.g., 540 degrees, although other wraps are also contemplated. The longer tube 102 wraps around multiple times to form a series of circular wraps either adjacent or partially overlapping, thereby arranged in a coil or spiral. The short tube 104 can be adjacent or overlapping the long tube 102. Note the lengths/wraps of the hoops 102, 104 illustrated are one example that can be utilized, it being understood that tubes 102 and 104 of lengths (and wraps) different than that illustrated and described are also contemplated. The short and long tubes could also be modified so that tube 104 is longer than tube 102.

Contained within the tubes 102, 104 are a delivery member 130 and a sheath 132 (FIG. 2) which are part of the delivery system of the implant. More specifically, the delivery member 130 is coupled to the implant and extends through the lumen of the sheath 132. The delivery sheath 132 and delivery member 130 with attached (coupled) implant are removed from the tubes 102, 104 for insertion of the implant into a patient’s body. Various embodiments of the delivery member 130 can be utilized and it can be indirectly coupled or directly coupled to the implant as shown for example in FIG. 39 of the ‘611 patent. The delivery member 130 can be in the form of a tube, a delivery wire or other configurations. Moreover, other types of delivery members could be provided to pull the implant 150 from the packaging container 101 and to advance the implant 150 into the body structure, e.g., the intracranial aneurysm.

The sheath 132 extends within the short tube 104 from a region adjacent the container 101, i.e., a region adjacent the opening at distal end 103 of short tube 104, and exits at the opposite (proximal) end 107 of short tube 104 through proximal opening 107a. A portion 133 of the sheath 132 is exposed in the gap 108 between the short and long tubes 104, 102, i.e., between proximal end 107 of short tube 104 and distal end 109 of long tube 102. The exposed portion 133 can be between about 1 cm and about 39 cm, although other dimensions are contemplated. The delivery member 130 extends through the lumen of the delivery sheath 132 and thus through the short tube 104 along with the sheath 132, thereby exiting proximal opening 107a of short tube 104 along with the sheath 132. The delivery member 130 extends out of the proximal opening at the proximal end 134 of sheath 132 such that portion 138 is exposed between the proximal end 134 of sheath 132 and the distal end 109 of long tube 102. The exposed portion 138 can be between about 1 cm and about 39 cm, although other dimensions are contemplated. Note exposed portion 138 is less than the total gap 108 since a portion of the delivery member 130 is within a portion of sheath 132 which is exposed in gap 108. The delivery member 130 extends through distal opening 135 at end 109 and through the lumen of the long tube 102. The delivery member 130 can terminate within the long tube 102 or alternatively can be of sufficient length to extend out of the opening at the proximal end 114 of long tube 102.

As can be appreciated, the vascular implant 150 is stored within the container 101 in its placement state (condition). That is, it is maintained in an unconstrained position (condition), corresponding to its secondary helical or complex shape, during packaging and shipping. This advantageously reduces the chances of mechanical creep (loss of shape recovery) of the implant 150, e.g., to take a set in a reduced diameter position or non-secondary shape which might occur if it were packaged within the delivery sheath itself since the delivery sheath has an internal diameter less than the internal diameter (dimension) of the bulbs 110 of container 101 and less than an outer diameter or transverse dimension of the unconstrained implant 150. In other words, the implant is maintained in the container in its secondary shape, that it is designed to be placed in the body, e.g., aneurysm, or at least at a shape closer to its placement shape than if it were in the smaller diameter delivery sheath, thus better ensuring it will return to this state after passage though the delivery sheath 132 and through the microcatheter into the body. If the implant 150 was shipped within the delivery sheath 132, it would be held in a constrained position with a reduced transverse dimension (reduced profile) in order to fit within the sheath since the sheath has a smaller diameter. Instead, the implant is retained outside the delivery sheath in a non-constrained or less constrained condition having a transverse cross-section greater than the transverse cross section or diameter of the sheath, until the point in time the implant is ready for insertion from the sheath into the catheter for insertion into the body. Packaging the implant in the 3D secondary configuration also allows the clinician to visually confirm the implant size and shape prior to insertion into the body. If it is packaged in a constrained condition within the sheath or packaging, the clinician would first need to remove the implant from the packaging to view its secondary shape, and then reload in into the packaging or sheath.

In other words, in the first condition within the container 101, the implant has a first transverse dimension. In the second condition when pulled within the delivery sheath, the implant has a second transverse dimension less than the first transverse dimension as it is straightened within the sheath. The first transverse dimension of the implant (when unconstrained) is greater than the transverse dimension, e.g., the diameter, of the delivery sheath. Note the transverse dimension can be considered as an overall height, perpendicular to a longitudinal axis of the implant. Thus, if a first imaginary line is drawn on a first side of the longitudinal axis tangent to the largest peak on the first side of the longitudinal axis of the implant and a second imaginary line is drawn tangent to the largest peak on the second opposite side of the longitudinal axis, a straight line distance connecting the two imaginary lines and drawn perpendicular to the longitudinal axis of the implant represents the transverse dimension. Note the transverse dimension is different than the primary diameter dimension of the implant. That is, it is the size of the overall shape of the implant that changes, i.e., it is moved from an unconstrained (or less constrained) condition in a non-linear shape (closer to or in its secondary shape) within the container to a constrained linear or more linear shape within the sheath (closer to or at its primary shape). In other words, bend radii of secondary shape loops of the implant are much greater in the constrained (sheathed) state than in the unconstrained or less constrained state.

It should be understood that when released from the delivery sheath 132 the implant will move to fill the body space - if the body space is smaller than the transverse dimension of the implant in its secondary shape then the implant will move to the dimension of the body space which is still larger than the transverse dimension of the delivery sheath.

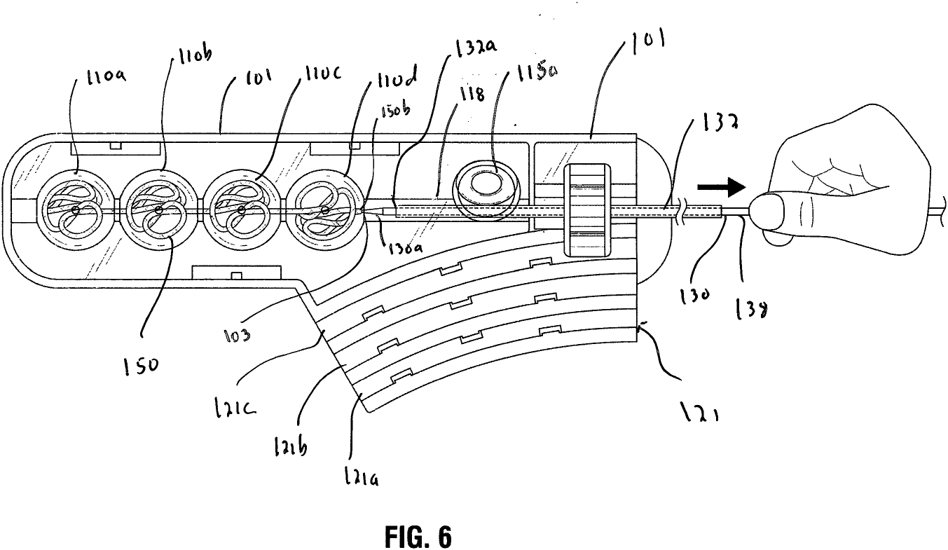

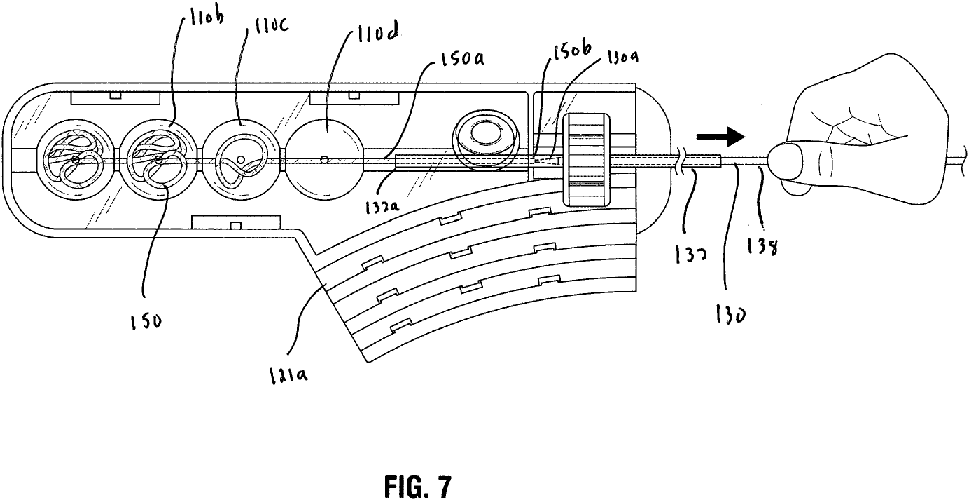

The method of loading the implant 150 from the container 101 into the sheath 132 for insertion into the microcatheter for delivery into the body lumen, e.g., vessel or aneurysm, of the patient will now be described. The method can be understood with reference to FIGS. 6-12. Note in FIGS. 7-10 the packaging tubes 102 and 104 have been removed for clarity but they would be positioned as shown in FIGS. 1 and 2.

In the first step (FIG. 6, the delivery member 130 is grasped at the exposed region (portion) 138 and at the sheath 132 by the clinician. As noted above, the exposed portion is between distal end 109 of long tube 102 and proximal end 134 of sheath 132 which extends beyond proximal end 107 of short tube 104. The distal end portion 130a of delivery member 130 extends distally of distal edge 132a of sheath 132. The clinician pulls the delivery member 130 relative to the sheath 132 in a direction (see arrow) away from the distal end of the short tube 104, (FIG. 2) e.g., in a proximal direction relative to the delivery sheath 132. The delivery member 130 can be attached to the implant at region 150b in various ways such as engagement of the cutout portions of the proximal tube as shown in FIGS. 37-40 and 41 of the ‘611 patent. By pulling the delivery member 130, the implant 150 is first pulled out of proximalmost bulb 110d, as shown in FIG. 7, and pulled into the sheath 132 through its distal opening at distal end 132a and into the lumen into a more elongated, e.g., more linear, position of reduced transverse dimension (constrained position). Note the implant 150 is straightened (see straightened region 150a) as it travels through channel 118 in which the delivery sheath 132 is seated.

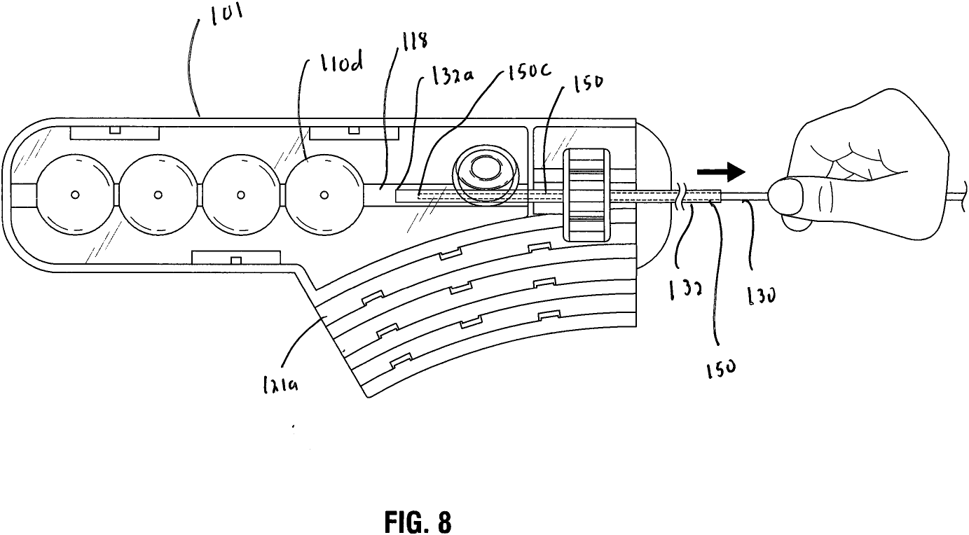

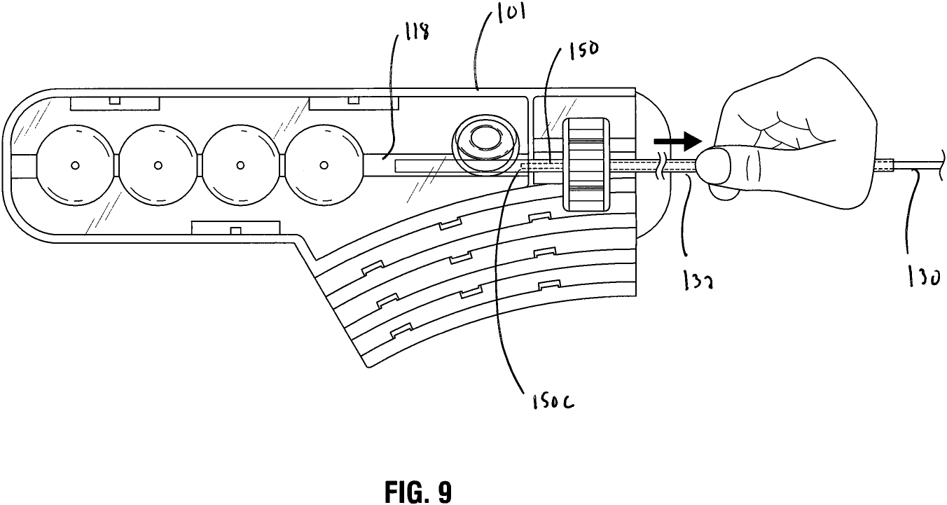

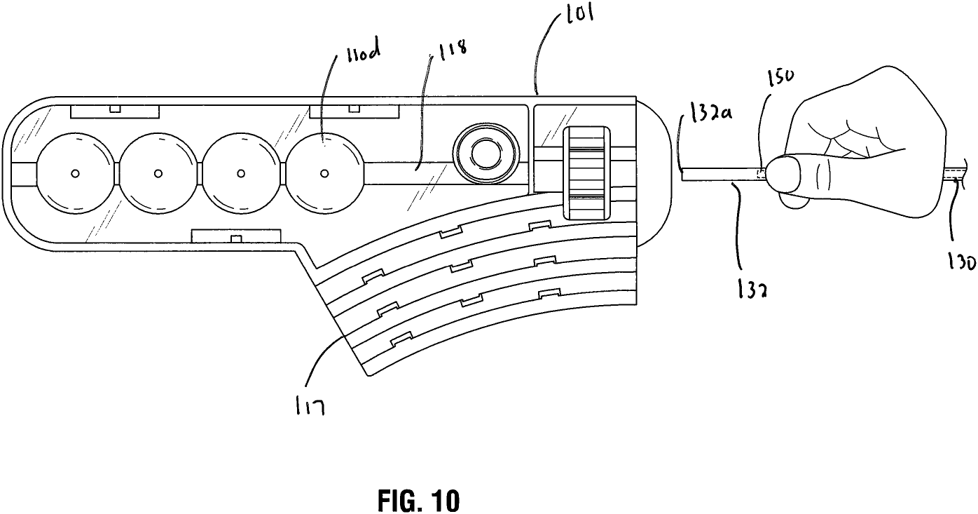

After the implant 150 is pulled from the container 101 and pulled fully inside the lumen of the sheath 132 (FIG. 8; See end 150c inside sheath 132), the clinician next (preferably with a single hand) grasps the exposed portion 133 of sheath 132 (FIG. 9), preferably at its end 134, and pulls it along with grasped delivery member 130 in a direction away from the short tube 104 (in the same direction the delivery member 130 was pulled in the previous step of FIG. 8, i.e., proximally as defined herein). After the sheath 132 and delivery member 130 have been pulled in the direction of the arrow of FIG. 9 and out of the opening 107 in the short tube 104, the distal end of the sheath 132 is now free (see FIG. 10) as the free (distal) end 132a of sheath 132 exposed from the short tube 104 of FIG. 2.

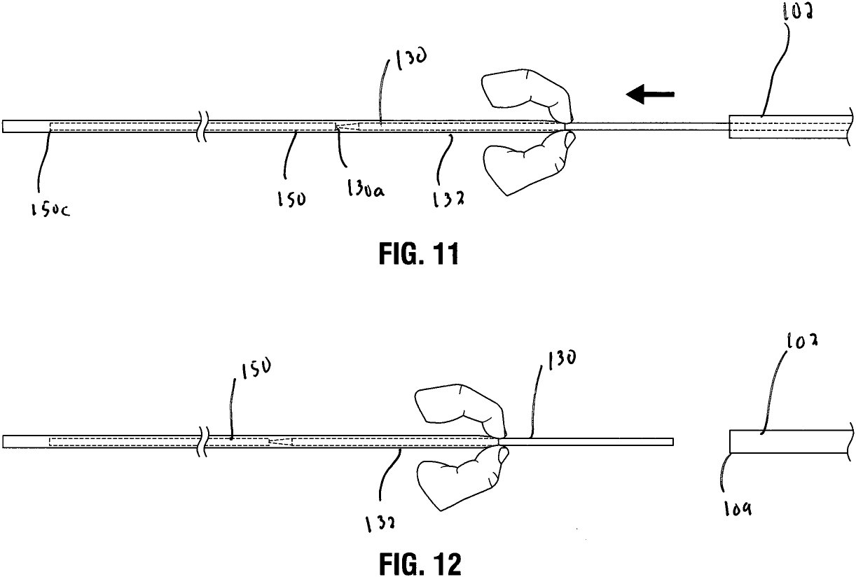

Next, the delivery member 130 is grasped by the clinician (FIG. 11) and the delivery member 130 is pulled in a direction away from the long tube 102. It is pulled in this direction of the arrow until the proximal end of the delivery member 130 is exposed from the end 109 of the long tube 102, thereby exposing a free end of delivery member 130 (see FIG. 12). As shown, the implant 150 is still contained within the delivery sheath 132. Now with the sheath 132 and delivery member 130 removed from the packaging hoops (tubes) 102, 104, the sheath 132 with internally contained delivery member 130 and coupled implant 150 can be inserted into or placed in abutment with a microcatheter for delivery of the implant 150 through the microcatheter and into the body lumen, i.e., intracranial aneurysm, in the methods described for example in the ‘611 patent. If placed in abutment, the delivery sheath distal end 132a would be slid through the microcatheter luer (hub) to abut the proximal end of the catheter and the RHV would be tightened to hold the delivery sheath in place and prevent the backflow of blood. Then the delivery member and coupled implant would be advanced through a lumen in the microcatheter, with the delivery sheath remaining outside the catheter.

In some embodiments, the delivery wire can be advanced until the proximal end is about 2 inches away from the proximal end of the delivery sheath. The RHV is loosened and the delivery sheath is removed proximally over the delivery wire. The delivery wire is then advanced to the desired site using fluoroscopic guidance. The marker bands on the microcatheter and delivery wire are then aligned and the implant can be detached from the delivery wire by mechanical decoupling or electrolytic detachment.

Note various attachments for the delivery member and implant can be utilized to pull the delivery member implant from the container and into the delivery sheath.

The delivery systems disclosed herein are for uses for delivering devices for treating intracranial aneurysms, however it is also contemplated that the delivery systems can be used to deliver devices through and in other body lumens in a patient.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure as defined by the claims appended hereto.

Although the systems, devices, apparatus and methods of the subject invention have been described with respect to preferred embodiments, those skilled in the art will readily appreciate that changes and modifications may be made thereto without departing from the spirit and scope of the present invention as defined by the appended claims.

Elements and features shown or described in connection with one embodiment may be combined with those of another embodiment without departing from the scope of the present invention.

Where a range of values is provided, it is understood that each intervening value between the upper and lower limit of that range and any other stated or intervening value in that stated range is encompassed by the present disclosure.

It should be noted that as used herein and in the appended claims, the singular forms “a”, “and”, and “the” include plural references unless the context clearly dictates otherwise.

Throughout the present disclosure, terms such as “approximately,” “generally,” “substantially,” and the like should be understood to allow for variations in any numerical range or concept with which they are associated. For example, it is intended that the use of terms such as “approximately”, “generally” and “substantially” should be understood to encompass variations on the order of 25% (e.g., to allow for manufacturing tolerances and/or deviations in design).

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present invention.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A packaging for a vascular implant, the packaging comprising a first packaging tube and a holder having at least one recess, the implant positioned within the holder in a first looped condition, and a looped portion of the implant includes a plurality of loops receivable in the at least one recess, wherein the implant is movable from the holder into the first packaging tube and maintained within the first packaging tube in a second more linear condition.

2. The packaging of claim 1, further comprising a delivery sheath having a lumen and a delivery member positioned within the lumen, the delivery sheath positioned within the first packaging tube, and the implant is pulled into the delivery sheath by the delivery member to be maintained within the delivery sheath within the first packaging tube.

3. The packaging of claim 1, further comprising a second packaging tube having a first end and a second opposite end, and the first packaging tube has a first end adjacent the holder and a second opposite end, the first end of the second packaging tube is spaced from the second end of the first packaging tube to form a gap between the first and second packaging tubes.

4. The packaging of claim 1, where the holder includes at least one arcuate channel to receive the first packaging tube in a lumen therein.

5. The packaging of claim 1, wherein the at least one recess comprises a plurality of spaced apart recesses.

6. The packaging of claim 5, wherein adjacent recesses are connected by an opening.

7. The packaging of claim 5, wherein the plurality of recesses are longitudinally aligned.

8. The packaging of claim 5, wherein each of the plurality of recesses is a same size.

9. The packaging of claim 5, wherein at least one of the plurality of recesses is a different size than another of the plurality of recesses.

10. The packaging of claim 2, wherein lateral movement of the delivery sheath is constrained within the holder by a friction element.

11. A packaging for a vascular implant comprising a container having a) a plurality of recesses each dimensioned to receive a looped portion of the implant, b) a channel, and c) a first member extending within the channel and engageable with an end region of the implant.

12. The packaging of claim 11, wherein the container comprises an arcuate channel to receive a portion of a packaging tube into which the implant is pulled.

13. The packaging of claim 12, wherein the packaging tube receives the first member.

14. The packaging of claim 11, wherein the plurality of recesses are longitudinally aligned.

15. The packaging of claim 11, wherein the recesses have covers to form bulbs.

16. The packaging of claim 15, wherein the bulbs are transparent.

17. The packaging of claim 11, wherein the plurality of recesses are substantially dome shaped.

18. The packaging of claim 11, wherein the plurality of recesses are connected by longitudinally extending recesses.

19. The system of claim 11, further comprising a first packaging tube and a second packaging tube, a first end of the second packaging tube is spaced from the second end of the first packaging tube to create a gap between the first and second packaging tubes, wherein an exposed portion of the first member is exposed within the gap between the first and second packaging tubes.

20. The system of claim 19, wherein application of a pulling force to the first member pulls the vascular implant from the container where it is held in an unconstrained condition into a delivery sheath so the vascular implant has a reduced transverse dimension.
